(19) United States
(12) Patent Application Publication
(10) Pub. No.: US 2009/0171212 A1
(43) Pub. Date: Jul. 2, 2009

(54) Interactive Virtual Visualization System For Training And Assistance In the Use of Ultrasound Handheld Transducers

(76) Inventor: Howard Mark Garon, Silver Spring, MD (US)

Correspondence Address: MIRUS TECHNOLOGIES, INC., 3819 GLEN EAGLES DRIVE, SILVER SPRING, MD, 20906, US

(21) Appl. No.: 11/869,735

(22) Filed: Oct. 9, 2007

(51) Int. Cl. A61B 8/14

(52) U.S. Cl. 600/446

(57) ABSTRACT

This invention provides a system and method for constructing and implementing an interactive virtual visualization system suitable for training and assisting an ultrasound operator to acquire useful images using a handheld transducer. The system requires an actual ultrasound machine complete with image display, controls and ultrasound transducer. As a direct result, skills obtained on the machine while in training mode do not have to be reacquired on a different machine in order for the operator to transit to live subjects. This also implies that the same machine may be used for refresher training. Two embodiments of the invention are presented, one in which the interactive virtualization system appears outboard to the ultrasound machine, the other in which the virtualization systems is embedded directed within the ultrasound machine.

### BACKGROUND OF INVENTION

Most ultrasound machines require that the operator properly manipulate a handheld transducer in order to gain a useful image. The operator must not only be familiar with the required motions but also the impact of those motions on acquiring a useful image. Ultrasound is the only imaging modality that remains almost completely dependent upon the operator of the machine. There are a few exceptions that provide some form of automated ultrasound image acquisition. Those automated exceptions usually accomplish the scan by in turn severely restricting the application. The new ultraportable ultrasound machines on the other hand have opened up entirely new clinical and field applications for ultrasound imaging. By their very nature, the new clinical and field applications are poor candidates for automata. The key to realizing the significant life-saving potential of these new applications, at least for the near future, will be to properly train all designated personnel in the use of the ultrasound machine with a handheld transducer.

Training the operator to develop a proper skillset and then to subsequently maintain that skillset has always been a difficult task. The trainee ordinarily begins with a phantom and then sequences to live subjects, usually fixating on a single application or protocol until a minimum skill level is achieved. The phantoms are of course severely restrictive when compared to live subjects. To partially compensate, some training systems substitute a special transducer and phantom. The transducer in these training systems is not a real functioning ultrasound transducer at all but contains an active device or devices for locating the transducer relative to the phantom. Once the relative position of the transducer is determined with respect to the phantom a stored image is then retrieved and displayed. The invention described here overcomes many of the limiting aspects of these training systems based upon the use of a phantom or pseudo-transducer and simultaneously permits the creation of considerably more diverse training scenarios. In one particular embodiment the primary application of the invention would be to train operators in conducting a human scan with an abdominal transducer. From an outside observer's viewpoint, the trainee appears to manipulate a conventional abdominal transducer over an acoustic slab consisting of a flat rectangular piece of polystyrene foam with dimensions 40 cm by 20 cm and approximately 1 cm thick. The trainee, however, is viewing the display of the ultrasound machine as though they were scanning a live human subject. When coupled to an ultrasound instrument augmented as described herein, this invention exploits the dimensions and acoustic properties of the slab in order to simulate any scanning protocol covering a limited portion of the human body. The acoustic slab's composition and form factor offers additional benefits conducive to acquiring skills both for the first time and for refreshing those skills. The slab is lightweight, flexible, easy to transport and relatively inexpensive to manufacture.

### SUMMARY OF INVENTION

This invention provides a system and method for constructing and implementing an interactive virtual visualization system suitable for training and assisting an ultrasound operator to acquire useful images using a handheld transducer. The ultrasound operator can also be taught certain scan protocols and how to interpret, in a limited fashion, the acquired images. The system requires an actual ultrasound machine complete with image display, controls and ultrasound transducer. This is a key advantage of the invention over prior art. Since a conventional ultrasound machine is used this allows the operator to immediately transit between training and actual application to live subjects. Skills obtained on the machine while in training mode do not have to be relearned on a machine with a totally different look and feel. This also implies that the invention can be used to refresh skills on a regular basis. The transducer may be either integral to the machine or attached to the machine via cable or wireless. In one embodiment of the invention, the ultrasound machine is required to have the capability of outputting acquired images in real-time or near real-time, or should be modifiable as such. An alternative realization provides for all the appropriate training software and interactive imaging to be embedded directly on the ultrasound machine.

### BRIEF DESCRIPTION OF THE DRAWINGS

FIG. 1 illustrates the general information flow for a dedicated ultrasound virtual visualization system.

FIG. 2 depicts how a conventional ultrasound system may be augmented with the virtual visualization system through the use of an operational mode selection switch 115. Switch 115 may be realized either in software or hardware. Elements 110 through 115 may be either external to the ultrasound machine or embedded within the machine. Finally,

FIG. 3 shows how the system may be further augmented to provide the operator with assistance in the scanning of live subjects.

### DETAILED DESCRIPTION OF THE INVENTION

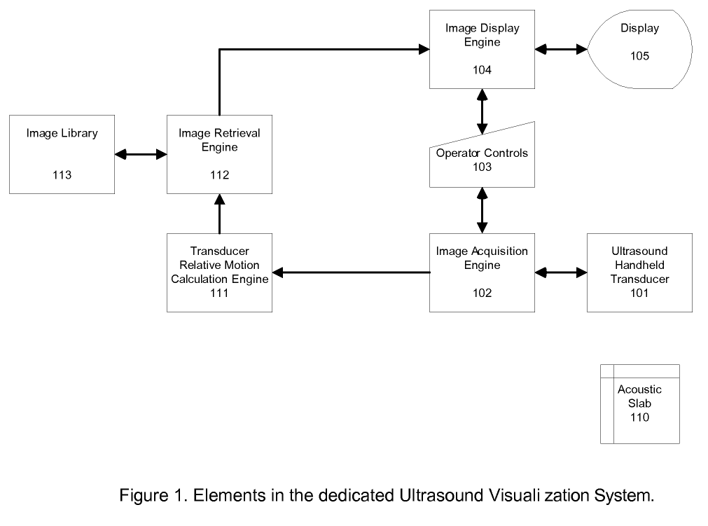

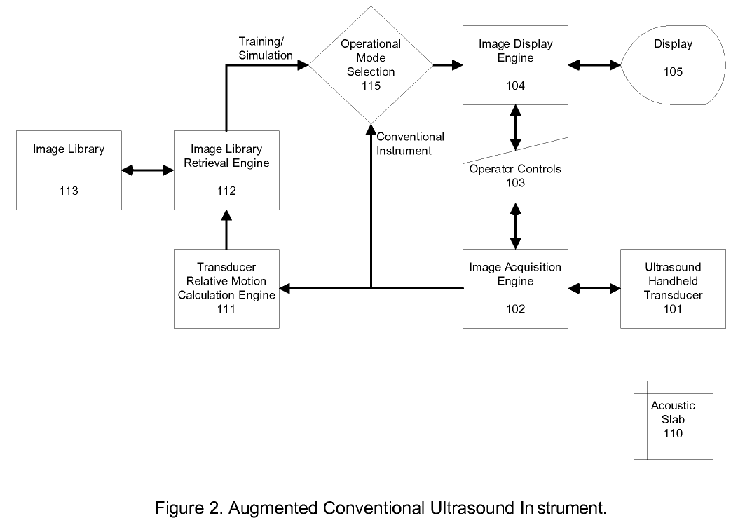

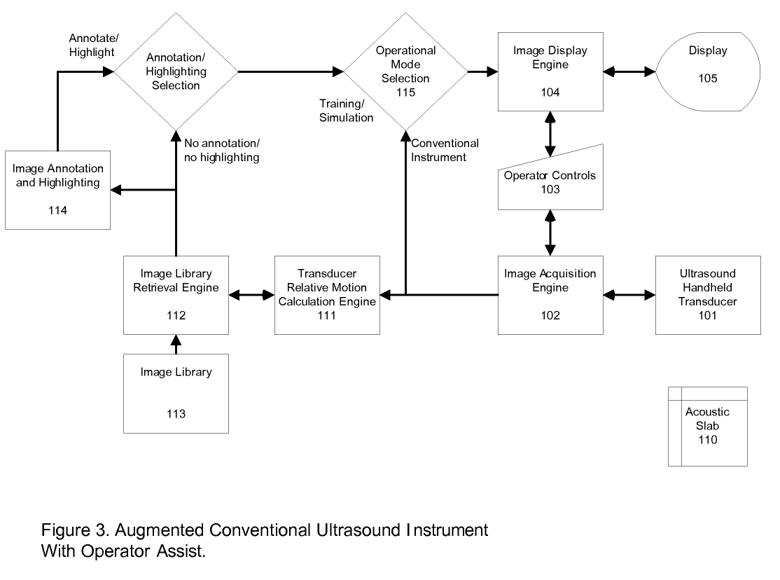

The preferred embodiment of the present invention is best understood by referring to FIGS. 1-3 of the drawings, like numerals being used for like and corresponding parts of the various drawings.

In all embodiments, an ultrasound transducer 101 is used to manually scan an acoustic slab 110. To obtain an optimum image a handheld transducer 101 is manipulated using one or a combination of three general motions—sweep, tilt and rotate. In the particular embodiment shown in FIG. 1 the transducer 101 is driven by and replies with image information to an image acquisition engine 102. The image acquisition engine then drives a transducer relative motion calculation engine 111. The output of the transducer relative motion calculation engine 111 in combination with operator controls 103 serve as a basis for image retrieval from the image library 113 using the image retrieval engine 112. The operator is able to view the output of the image retrieval engine 112 through the image display engine 104 on the display 105. The complete process, from transducer image acquisition through operator display, may be accomplished in real time.

While the acoustic slab 101 is depicted as a rectangular slab, it may in fact assume any three dimensional shape (i.e., having volume). Depending upon the training application, some volumetric shapes will be more appropriate than others. A rectangular volumetric slab is depicted here for simplicity in demonstration. The slab may have embedded specular targets distributed randomly, uniformly or with some specified volumetric pattern throughout the slab. The specular targets could even be generated simply by taking advantage of ordinary manufacturing defects found in polystyrene foam packing material. An acoustic map that delineates the spatial distribution of all targets and/or defects across the surface of the slab would then enable a unique determination of surface position relative to a fixed reference on the slab by image differencing. This acoustic map would be stored and maintained as an integral part of the transducer relative motion calculation engine 111.

The interaction between transducer and slab and its subsequent virtual visualization will enable each trainee to acquire the eye-hand coordination necessary towards obtaining optimum ultrasound images. Training may be further enhanced by employing acoustically transparent plastic overlays either over the acoustic slab or, in the case of an optically transparent or translucent acoustic slab, beneath or within the interior of the slab. For instance, if the goal of the training relates to human physiology and pathology, the plastic overlays could have imprinted the surface anatomical features of a specific section of the body. This would provide the trainee with a sense of subject orientation with respect to transducer motion. The complete system would then continually prompt the trainee as to the proper handling of the transducer in order to achieve the goal or goals of the syllabus. With this interactive virtual system a syllabus can be constructed to teach almost any protocol. All that is required is to maintain an adequate image database corresponding to the case pathologies that are to be introduced to the trainee along with corresponding overlays of the acoustic slab.

The specific examples and embodiments given above are not intended to limit the application of the invention but rather to aid in understanding its construction.

The invention claimed is:

1. An interactive virtual visualization system suitable for training and assisting an ultrasound operator in the art of acquiring images using a handheld transducer, such system comprised of:
an ultrasound machine capable of outputting captured images;
an acoustic slab with specular targets and/or defects whose unique relative positions between targets and/or defects in turn allow the calculation of the motion between captured ultrasound images;
a processing unit intended to augment the ultrasound machine and provide, upon input of the captured images, virtual visual output consistent with the motion of the handheld transducer across the acoustic slab.

2. The system of claim 1, further comprising a library of digitally stored images, such images representing the historical output of another or the same ultrasound machine and which in turn maps the entire surface range of transducer motion over the height and width of the acoustic slab with a predetermined spatial sampling interval between image samples.

3. The system of claim 1, further comprising a special processing unit to match relative or absolute motion of the transducer to select images from within the digitally stored library of images.

4. The system of claim 1, wherein the virtual visualization processing unit, library image selection processor, library of digitally stored images and ultrasound machine are all contained within a single enclosure.

5. The method wherein the absolute position, relative position and motion of the ultrasound transducer is determined with respect to the acoustic slab.

6. The system and method whereby an acoustically transparent overlay is imprinted with simulated surface features, such features serving to represent the surface of the object or human subject of particular interest in a given training session, and placed over, beneath or inside the acoustic slab of claim 1.
